(19) United States
(12) Patent Application Publication
(10) Pub. No.: US 2023/0111299 A1
(43) Pub. Date: Apr. 13, 2023

(54) SYSTEM FOR AIR VOLUME CORRECTION BASED ON FLUID PRESSURE AND FLOW RATE

(71) Applicant: BAYER HEALTHCARE LLC, WHIPPANY, NJ (US)

(72) Inventors: MICHAEL MCDERMOTT, BERLIN (DE); WILLIAM BARONE, PITTSBURGH, PA (US); RONALD BROWN, WEST NEWTON, PA (US); TIM NEWING, THORNLEIGH (AU)

(21) Appl. No.: 17/913,202

(22) PCT Filed: Mar. 29, 2021
(86) PCT No.: PCT/US2021/024625

§ 371 Date: Sep. 21, 2022

Related U.S. Application Data
(60) Provisional application No. 63/002,885, filed on Mar. 31, 2020.

(51) Int. Cl. A61M 5/00; A61M 5/168

(52) U.S. Cl. CPC A61M 5/007; A61M 5/16827

(57) ABSTRACT

A method for determining a volume of one or more air bubbles in a fluid path includes initiating an injection procedure in which at least one medical fluid is injected into the fluid path, receiving an electrical signal from an air detector of the fluid injector system, wherein the electrical signal indicates the presence of one or more air bubbles in the fluid path, calculating a flow rate of fluid in the fluid path, determining a fluid pressure in the fluid path, determining a count value of the one or more air bubbles representative of a volume of the one or more air bubbles, and updating a cumulative counter with the count value of the one or more air bubbles. The cumulative counter is representative of a cumulative volume of air that has passed through the fluid path during the injection procedure.

### CROSS-REFERENCE TO RELATED APPLICATIONS

This application claims the benefit of U.S. Provisional Patent Application No. 63/002,885 filed on Mar. 31, 2021, the disclosure of which is hereby incorporated by reference.

### BACKGROUND OF THE DISCLOSURE

### Field of the Disclosure

The present disclosure relates generally to detection of air during a medical fluid injection. More particularly, the present disclosure relates to a method, system, and computer program product for determining a volume of one or more air bubbles in a fluid path of a fluid injector system.

### Description of Related Art

In many medical diagnostic and therapeutic procedures, a medical practitioner, such as a physician, injects a patient with one or more medical fluids. In recent years, a number of injector-actuated syringes and powered fluid injectors for pressurized injection of medical fluids, such as a contrast solution (often referred to simply as “contrast”), a flushing agent (such as saline or Ringer's lactate), and other medical fluids, have been developed for use in procedures such as cardiovascular angiography (CV), computed tomography (CT), ultrasound, magnetic resonance imaging (MRI), positron emission tomography (PET), and other imaging procedures. In general, these fluid injectors are designed to deliver a preset amount of fluid at a preset pressure and/or flow rate.

Typically, fluid injectors have at least one drive member, such as a piston, that connects to the syringe, for example via connection with a plunger or an engagement feature on a proximal end wall of the syringe. The syringe may include a rigid barrel with the syringe plunger being slidably disposed within the barrel. The drive members drive the plungers in a proximal and/or distal direction relative to a longitudinal axis of the barrel to draw fluid into or deliver the fluid from the syringe barrel. Alternatively, the fluid injector may include a drive member for driving rotating a peristaltic pump to pump a medical fluid through a tubing and deliver the fluid to a patient.

Prior to injecting fluid into the patient, the fluid injectors are purged of air using various methods. However, due to various characteristics of the components of fluid injectors and the complex nature of fluid flow, small amounts of air may remain in components of the fluid injector even after purging operations are performed. Injection of air or other gas bubbles, especially during high pressure injections, can cause patient harm and should be avoided. In addition to potentially harming the patient, air bubbles can also manifest as artifacts in reconstructed images of the patient's vasculature, which can interference with diagnosis. To these ends, some existing fluid injectors are capable of detecting air bubbles in various fluid paths and aborting an injection procedure in response to the detection of such air bubbles.

However, for some injection procedures such as CT, small volumes of air may present minimal threat to patient safety and aborting an injection after air detection of a safe volume of air bubbles is undesirable. However, existing fluid injectors which are incapable of accurately determining the volume of air bubbles in the system must take a conservative approach to air bubble detection; and may thus unnecessarily abort injections which pose no clinical threat to the patient. Unnecessarily aborting an injection may interrupt workflow, reduce patient and clinician confidence and satisfaction with the procedure, and/or result in additional radiation/contrast media exposure for the patient as the injection must be restarted.

### SUMMARY OF THE DISCLOSURE

In view of the foregoing, there exists a need for methods, systems, and computer program products for detecting air bubbles and accurately determining the volume of air present in fluid paths of fluid injector systems during a medical fluid injection procedure such as a contrast enhanced imaging procedure. In view of these needs, embodiments of the present disclosure are directed to a method for determining a volume of one or more air bubbles in a fluid path of a fluid injector system. In some embodiments, the method includes initiating an injection procedure in which at least one medical fluid is injected into the fluid path and receiving an electrical signal from an air detector of the fluid injector system. The electrical signal indicates the presence of one or more air bubbles in the fluid path. The method further includes calculating a flow rate of fluid in the fluid path, determining a fluid pressure in the fluid path, and determining a count value of the one or more air bubbles based on a duration for which the electrical signal is received, the flow rate, and the fluid pressure. The count value is representative of a volume of the one or more air bubbles. The method further includes updating a cumulative counter with the count value of the one or more air bubbles, wherein the cumulative counter is representative of a cumulative volume of air that has passed through the fluid path during the injection procedure.

In some embodiments, the method further includes halting the injection procedure in response to the cumulative counter exceeding a predetermined threshold.

In some embodiments, the method further includes continuing the injection procedure in response to the cumulative counter being below a predetermined threshold.

In some embodiments, the predetermined threshold is programmed into a memory of the fluid injector system.

In some embodiments, calculating the flow rate in the fluid path includes estimating an actual flow rate in the fluid path based on a commanded flow rate for the injection procedure and compliance of one or more components of the fluid injector system.

In some embodiments, the method further includes setting the cumulative counter to zero prior to initiating the injection procedure.

In some embodiments, the method further includes purging one or more air bubbles from the fluid injector system prior to initiating the injection procedure.

Other embodiments of the present disclosure are directed to a fluid injector system including at least one syringe configured for injecting at least one medical fluid, a fluid path in fluid communication with the at least one syringe, an air detector configured to detect one or more air bubbles in the fluid path, and at least one processor. The at least one processor is programmed or configured to initiate an injection procedure in which the at least one medical fluid is injected from the at least one syringe into the fluid path, receive an electrical signal from the air detector, wherein the electrical signal indicates the presence of one or more air bubbles in the fluid path, calculate a flow rate of fluid in the fluid path, determine a fluid pressure in the fluid path, and determine a count value of the one or more air bubbles based on a duration for which the electrical signal is received, the flow rate, and the fluid pressure. The count value is representative of a volume of the one or more air bubbles. The at least one processor is further programmed or configured to update a cumulative counter with the count value of the one or more air bubbles. The cumulative counter is representative of a cumulative volume of air that has passed through the fluid path during the injection procedure.

In some embodiments, the at least one processor is further programmed or configured to halt the injection procedure in response to the cumulative counter exceeding a predetermined threshold.

In some embodiments, the at least one processor is further programmed or configured to continue the injection procedure in response to the cumulative counter being below a predetermined threshold.

In some embodiments, the predetermined threshold is programmed into a memory of the fluid injector system.

In some embodiments, calculating the flow rate in the fluid path includes estimating an actual flow rate in the fluid path based on a commanded flow rate for the injection procedure and compliance of one or more components of the fluid injector system.

In some embodiments, the at least one processor is further programmed or configured to set the cumulative counter to zero prior to initiating the injection procedure.

In some embodiments, the at least one processor is further programmed or configured to purge one or more air bubbles from the fluid injector system prior to initiating the injection procedure.

Other embodiments of the present disclosure are directed to a computer program product for determining a volume of one or more air bubbles in a fluid path of a fluid injector system. The computer program product includes non-transitory computer readable media including one or more instructions that, when executed by at least one processor of the fluid injector system, cause the at least one processor to initiate an injection procedure in which at least one medical fluid is injected into the fluid path and receive an electrical signal from an air detector of the fluid injector system. The electrical signal indicates the presence of one or more air bubbles in the fluid path. The one or more instructions further cause the at least one processor to calculate a flow rate of fluid in the fluid path, determine a fluid pressure in the fluid path, and determine a count value of the one or more air bubbles based on a duration for which the electrical signal is received, the flow rate, and the fluid pressure. The count value is representative of a volume of the one or more air bubbles. The one or more instructions further cause the at least one processor to update a cumulative counter with the count value of the one or more air bubbles. The cumulative counter is representative of a cumulative volume of air that has passed through the fluid path during the injection procedure.

In some embodiments, the one or more instructions further cause the at least one processor to halt the injection procedure in response to the cumulative counter exceeding a predetermined threshold.

In some embodiments, the one or more instructions further cause the at least one processor to continue the injection procedure in response to the cumulative counter being below a predetermined threshold.

In some embodiments, the predetermined threshold is programmed into a memory of the fluid injector system.

In some embodiments, calculating the flow rate in the fluid path includes estimating an actual flow rate in the fluid path based on a commanded flow rate for the injection procedure and compliance of one or more components of the fluid injector system.

In some embodiments, the one or more instructions further cause the at least one processor to set the cumulative counter to zero prior to initiating the injection procedure.

In some embodiments, the one or more instructions further cause the at least one processor to purge one or more air bubbles from the fluid injector system prior to initiating the injection procedure.

Further aspects or examples of the present disclosure are described in the following numbered clauses:

Clause 1. A method for determining a volume of one or more air bubbles in a fluid path of a fluid injector system, the method comprising: initiating an injection procedure in which at least one medical fluid is injected into the fluid path; receiving an electrical signal from an air detector of the fluid injector system, wherein the electrical signal indicates the presence of one or more air bubbles in the fluid path; calculating a flow rate of fluid in the fluid path; determining a fluid pressure in the fluid path; determining a count value of the one or more air bubbles based on a duration for which the electrical signal is received, the flow rate, and the fluid pressure, wherein the count value is representative of a volume of the one or more air bubbles; and updating a cumulative counter with the count value of the one or more air bubbles, wherein the cumulative counter is representative of a cumulative volume of air that has passed through the fluid path during the injection procedure.

Clause 2. The method of clause 1, further comprising: halting the injection procedure in response to the cumulative counter exceeding a predetermined threshold.

Clause 3. The method of clause 1 or 2, further comprising: continuing the injection procedure in response to the cumulative counter being below a predetermined threshold.

Clause 4. The method of any of clauses 1-3, wherein the predetermined threshold is programmed into a memory of the fluid injector system.

Clause 5. The method of any of clauses 1-4, wherein calculating the flow rate in the fluid path comprises estimating an actual flow rate in the fluid path based on: a commanded flow rate for the injection procedure; and compliance of one or more components of the fluid injector system.

Clause 6. The method of any of clauses 1-5, further comprising: setting the cumulative counter to zero prior to initiating the injection procedure.

Clause 7. The method of any of clauses 1-6, further comprising: purging one or more air bubbles from the fluid injector system prior to initiating the injection procedure.

Clause 8. A fluid injector system comprising: at least one syringe configured for injecting at least one medical fluid; a fluid path in fluid communication with the at least one syringe; an air detector configured to detect one or more air bubbles in the fluid path; at least one processor programmed or configured to: initiate an injection procedure in which the at least one medical fluid is injected from the at least one syringe into the fluid path; receive an electrical signal from the air detector, wherein the electrical signal indicates the presence of one or more air bubbles in the fluid path; calculate a flow rate of fluid in the fluid path; determine a fluid pressure in the fluid path; determine a count value of the one or more air bubbles based on a duration for which the electrical signal is received, the flow rate, and the fluid pressure, wherein the count value is representative of a volume of the one or more air bubbles; and update a cumulative counter with the count value of the one or more air bubbles, wherein the cumulative counter is representative of a cumulative volume of air that has passed through the fluid path during the injection procedure.

Clause 9. The fluid injector system of clause 8, wherein the at least one processor is further programmed or configured to: halt the injection procedure in response to the cumulative counter exceeding a predetermined threshold.

Clause 10. The fluid injector system of clause 8 or 9, wherein the at least one processor is further programmed or configured to: continue the injection procedure in response to the cumulative counter being below a predetermined threshold.

Clause 11. The fluid injector system of any of clauses 8-10, wherein the predetermined threshold is programmed into a memory of the fluid injector system.

Clause 12. The fluid injector system of any of clauses 8 to 11, wherein calculating the flow rate in the fluid path comprises estimating an actual flow rate in the fluid path based on: a commanded flow rate for the injection procedure; and compliance of one or more components of the fluid injector system.

Clause 13. The fluid injector system of any of clauses 8-12, wherein the at least one processor is further programmed or configured to: set the cumulative counter to zero prior to initiating the injection procedure.

Clause 14. The fluid injector system of any of clauses 8-13, wherein the at least one processor is further programmed or configured to: purge one or more air bubbles from the fluid injector system prior to initiating the injection procedure.

Clause 15. A computer program product for determining a volume of one or more air bubbles in a fluid path of a fluid injector system, the computer program product comprising: non-transitory computer readable media comprising one or more instructions that, when executed by at least one processor of the fluid injector system, cause the at least one processor to: initiate an injection procedure in which at least one medical fluid is injected into the fluid path; receive an electrical signal from an air detector of the fluid injector system, wherein the electrical signal indicates the presence of one or more air bubbles in the fluid path; calculate a flow rate of fluid in the fluid path; determine a fluid pressure in the fluid path; determine a count value of the one or more air bubbles based on a duration for which the electrical signal is received, the flow rate, and the fluid pressure, wherein the count value is representative of a volume of the one or more air bubbles; and update a cumulative counter with the count value of the one or more air bubbles, wherein the cumulative counter is representative of a cumulative volume of air that has passed through the fluid path during the injection procedure.

Clause 16. The computer program product of clause 15, wherein the one or more instructions further cause the at least one processor to: halt the injection procedure in response to the cumulative counter exceeding a predetermined threshold.

Clause 17. The computer program product of clause 15 Or 16, wherein the one or more instructions further cause the at least one processor to: continue the injection procedure in response to the cumulative counter being below a predetermined threshold.

Clause 18. The computer program product of any of clauses 15-17, wherein the predetermined threshold is programmed into a memory of the fluid injector system.

Clause 19. The computer program product of any of clauses 15-18, wherein calculating the flow rate in the fluid path comprises estimating an actual flow rate in the fluid path based on: a commanded flow rate for the injection procedure; and compliance of one or more components of the fluid injector system.

Clause 20. The computer program product of any of clauses 15-19, wherein the one or more instructions further cause the at least one processor to: set the cumulative counter to zero prior to initiating the injection procedure.

Clause 21. The computer program product of any of clauses 15-20, wherein the one or more instructions further cause the at least one processor to: purge one or more air bubbles from the fluid injector system prior to initiating the injection procedure.

Further details and advantages of the various examples described in detail herein will become clear upon reviewing the following detailed description of the various examples in conjunction with the accompanying drawing figures.

### BRIEF DESCRIPTION OF THE DRAWINGS

FIG. 1 is a perspective view of a fluid injector system according to an embodiment of the present disclosure;

FIG. 2 is a perspective view of a multi-use disposable set for use with the fluid injector system of FIG. 1;

FIG. 3 is a schematic view of various fluid paths within the fluid injector system of FIG. 1;

FIG. 4 is a schematic diagram of an electronic controller of the fluid injector system of FIG. 1;

FIG. 5A is a reconstructed image from an experimental injection performed with the fluid injector system of FIG. 1;

FIG. 5B is a reconstructed image from an experimental injection performed with the fluid injector system of FIG. 1, in which air bubbles were co-injected with the medical fluid;

FIG. 5C is a reconstructed image from an experimental injection performed with the fluid injector system of FIG. 1, in which air bubbles were co-injected with the medical fluid;

FIG. 6 is a schematic of an outlet air detector of the fluid injector system of FIG. 1;

FIG. 7 is a graph of an output electrical signal from the air detector of FIG. 6 in accordance with an embodiment of the present disclosure;

FIG. 8 is sequence diagram of a method for determining a volume of one or more air bubbles in a fluid path, according to an embodiment of the present disclosure;

FIG. 9 is a graph illustrating commanded flow rate and actual flow rate over time for an exemplary injection procedure;

FIG. 10 is a graph illustrating experimental and modeled flow rate data over time for an exemplary injection procedure;

FIG. 11 is a sequence diagram of a method for detecting air during setup and performance of an injection procedure, according to an embodiment of the present disclosure;

FIG. 12 is a graph illustrating an example data set of plunger displacement and pressure values during pressurization of the syringes of the fluid injector system of FIG. 1; and

FIG. 13 is a graph illustrating a compliance compensation equation for calculating air volume in the syringes of the fluid injector system of FIG. 1.

Referring to the drawings in which like reference characters refer to like parts throughout the several views thereof, the present disclosure is generally directed to an in-line air bubble suspension apparatus for use with an angiography injector system.

### DETAILED DESCRIPTION

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. Spatial or directional terms, such as “left”, “right”, “inner”, “outer”, “above”, “below”, and the like, are not to be considered as limiting as the invention can assume various alternative orientations.

As used herein, the singular form of “a”, “an”, and “the” include plural referents unless the context clearly dictates otherwise.

All numbers used in the specification and claims are to be understood as being modified in all instances by the term “about”. The terms “approximately”, “about”, and “substantially” mean a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

It is also to be understood that the specific devices and processes illustrated in the attached drawings, and described in the following specification, are simply exemplary examples of the disclosure. Hence, specific dimensions and other physical characteristics related to the examples disclosed herein are not to be considered as limiting.

When used in relation to a component of a fluid delivery system such as a fluid reservoir, a syringe, or a fluid line, the term “distal” refers to a portion of said component nearest to a patient. When used in relation to a component of a injector system such as a fluid reservoir, a syringe, or a fluid line, the term “proximal” refers to a portion of said component nearest to the injector of the injector system (i.e. the portion of said component farthest from the patient). When used in relation to a component of a fluid delivery system such as a fluid reservoir, a syringe, or a fluid line, the term “upstream” refers to a direction away from the patient and towards the injector of the injector system. For example, if a first component is referred to as being “upstream” of a second component, the first component is located nearer to the injector than the second component is to the injector. When used in relation to a component of a fluid delivery system such as a fluid reservoir, a syringe, or a fluid line, the term “downstream” refers to a direction towards the patient and away from the injector of the fluid delivery system. For example, if a first component is referred to as being “downstream” of a second component, the first component is located nearer to the patient than the second component is to the patient.

As used herein, the terms “capacitance” and “compliance” are used interchangeably to refer to a volumetric expansion of injector components, such as fluid reservoirs, syringes, fluid lines, and/or other components of a fluid delivery system as a result of pressurized fluids with such components and/or uptake of mechanical slack by force applied to components. Capacitance and compliance may be due to high injection pressures, which may be on the order of up to 325 psi in certain CT procedures and up to 1200 psi in some angiographic procedures, and may result in a volume of fluid held within a portion of a component in excess of the desired quantity selected for the injection procedure or the resting volume of the component. Additionally, capacitance of various components can, if not properly accounted for, adversely affect the accuracy of pressure sensors of the injector system because the volumetric expansion of components can cause an artificial drop in measured pressure of those components.

The terms “first”, “second”, and the like are not intended to refer to any particular order or chronology, but refer to different conditions, properties, or elements.

All documents referred to herein are “incorporated by reference” in their entirety.

The term “at least” is synonymous with “greater than or equal to”. The term “not greater than” is synonymous with “less than or equal to”.

It is to be understood that the disclosure may assume alternative variations and step sequences, except where expressly specified to the contrary. It is also to be understood that the specific devices and processes illustrated in the attached drawings, and described in the following specification, are simply exemplary aspects of the disclosure. Hence, specific dimensions and other physical characteristics related to the examples disclosed herein are not to be considered as limiting.

While the systems and apparatuses described herein are with reference to computed tomography (CT) injection systems, other pressurized injection protocols, such as an angiography (CV), positron emission tomography (PET), and magnetic resonance imaging (MRI) may also incorporate the various embodiments described herein for determining air bubble volume and preventing injection of air during an injection procedure.

Referring to the drawings in which like reference characters refer to like parts throughout the several views thereof, the present disclosure is generally directed to fluid injector systems having features for detection of air bubbles and preventing unsafe injections of air to the patient. The present disclosure is further directed to methods and computer-program products for accurately determining the volume of air bubbles and controlling various operations of the fluid injector system based on such determinations.

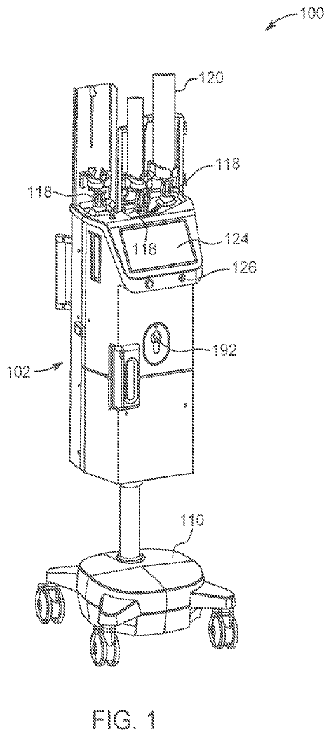

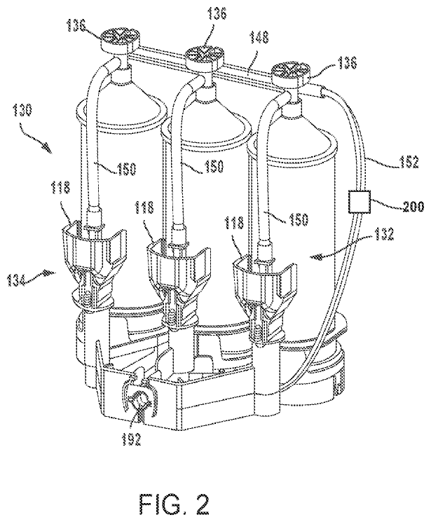

Referring first to FIGS. 1-3, an embodiment of the present disclosure is generally directed to a multi-fluid medical injector/injector system 100 (hereinafter “fluid injector system 100”, see FIG. 1) which in certain embodiments or aspects may include a multi-use disposable set (MUDS) 130 (see, FIG. 2) configured for delivering fluid to a patient using a single-use disposable set (SUDS) 190 connector, and in other embodiments or aspects may include two or more disposable fluid reservoirs or syringes 132, which may be disposed after one injection procedure or a specific number of injection procedures. The fluid injector system 100 may be a piston driven, syringe-based fluid delivery system and may include multiple components as individually described herein. Generally, the fluid injector system 100 illustrated in FIGS. 1-3 has a powered fluid injector or other administration device and a fluid delivery set intended to be associated with the powered fluid injector to deliver one or more fluids from one or more multi-dose containers under pressure into a patient, as described herein. The various devices, components, and features of the fluid injector system 100 and the fluid delivery set associated therewith are likewise described in detail herein.

While the various examples of the methods and processes of the present disclosure are described herein with reference to the fluid injector system 100 having the MUDS 130 and the SUDS 190 configuration of FIGS. 1-3, the disclosure is not limited to such an injector system and may be utilized in other syringe based injector systems, such as but not limited to those described in U.S. Pat. Nos. 7,553,294; 7,563,249; 8,945,051; 9,173,995; 10,124,110; 10,507,319; 10,583,256; and U.S. Application Publication No. 2018/0161496, the disclosures of each of which are incorporated herein in their entirety by this reference. Examples of fluid injector system 100 include the MEDRAD® Centargo CT Injection System available from Bayer HealthCare LLC.

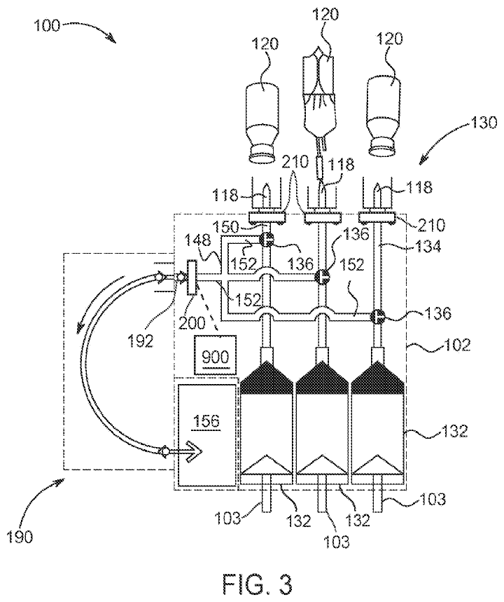

With reference to FIG. 1, the fluid injector system 100 according to one example includes an injector housing 102 that encloses the various mechanical drive components, electrical and power components necessary to drive the mechanical drive components, and control components, such as electronic memory and electronic control devices, used to control operation of reciprocally movable pistons 103 (see FIG. 3) associated with the fluid injector system 100 described herein. Such pistons 103 may be reciprocally operable via electro-mechanical drive components such as a ball screw shaft driven by a motor, a voice coil actuator, a rack-and-pinion gear drive, a linear motor, and the like.

The fluid injector system 100 may include at least one bulk fluid connector 118 for connection with at least one bulk fluid source 120. In some examples, a plurality of bulk fluid connectors 118 may be provided. For example, as shown in the fluid injector embodiment illustrated in FIG. 1, three bulk fluid connectors 118 may be provided in a side-by-side or other arrangement. In some examples, the at least one bulk fluid connector 118 may include a spike configured for removably connecting to the at least one bulk fluid source 120, such as a vial, a bottle, or a bag. The at least one bulk fluid connector 118 may be fluidly connected to the MUDS 130 (shown in FIG. 2), as described herein. The at least one bulk fluid source 120 may be configured for receiving a medical fluid, such as saline, Ringer's lactate, an imaging contrast medium solution, or other medical fluid, for delivery by the fluid injector system 100.

With reference to FIGS. 2 and 3, the MUDS 130 is configured for being removably connected to the fluid injector system 100 for delivering one or more fluids from the one or more bulk fluid sources 120 to the patient. Examples and features of embodiments of the MUDS 130 are further described in PCT International Publication No. WO 2016/112163, the disclosure of which is incorporated herein by reference in its entirety. The MUDS 130 may include one or more fluid reservoirs, such as one or more syringes 132. As used herein, the term “fluid reservoir” means any container capable of taking in and delivering a fluid, for example during a fluid injection procedure including, for example a syringe, a rolling diaphragm, a pump, a peristaltic pump, a compressible bag, and the like. Fluid reservoirs may include the interior volume of at least a portion of a fluid pathway, such as one or more tubing lengths, that are in fluid communication with the interior of the fluid reservoir, including fluid pathway portions that remain in fluid communication with the fluid reservoir after the system is closed or fluidly isolated from the remainder of the fluid pathway. In some examples, the number of fluid reservoirs may correspond to the number of bulk fluid sources 120 (shown in FIG. 1). For example, with reference to FIG. 2, the MUDS 130 has three syringes 132 in a side-by-side arrangement such that each syringe 132 is fluidly connectable to one or more of the corresponding three bulk fluid sources 120. In some examples, one or more bulk fluid sources 120 may be connected to one or more syringes 132 of the MUDS 130. Each syringe 132 may be fluidly connectable to one of the bulk fluid sources 120 by a corresponding bulk fluid connector 118 and an associated MUDS fluid path 134. The MUDS fluid path 134 may have a spike element that connects to the bulk fluid connector 118 and the fluid line 150. In some examples, the bulk fluid connector 118 may be provided directly on the MUDS 130.

With continued reference to FIGS. 1-3, the MUDS 130 may include one or more valves 136, such as stopcock valves, for controlling which medical fluid or combinations of medical fluids are withdrawn from the multi-dose bulk fluid source 120 (see FIG. 1) into the fluid reservoirs 132 and/or are delivered to a patient from each fluid reservoir 132. In some examples, the one or more valves 136 may be provided on a distal end of the plurality of syringes 132 or on a manifold 148. The manifold 148 may be in selectable fluid communication via the valves 136 with the interior volume of each of the syringes 132. The interior volume of the syringes 132 may be in selectable fluid communication via valves 136 with a first end of the MUDS fluid path 134 that connects each syringe 132 to the corresponding bulk fluid source 120. The opposing second end of the MUDS fluid path 134 may be connected to the respective bulk fluid connector 118 that is configured for fluidly connecting with the bulk fluid source 120. Depending on the position of the one or more valves 136, fluid may be drawn into the interior volume of the one or more syringes 132 or it may be delivered from the interior volume of the one or more syringes 132 to the patient via the manifold 148 and a fluid outlet line 152. In a first position, such as during the filling of the syringes 132, the one or more valves 136 are oriented such that fluid flows from the bulk fluid source 120 into the desired syringe 132 through a fluid inlet line 150, such as a MUDS fluid path 134. During the filling procedure, the one or more valves 136 are positioned such that fluid flow through one or more fluid outlet lines 152 and/or the manifold 148 is blocked or closed. In a second position, such as during a fluid delivery procedure, fluid from one or more syringes 132 is delivered to the manifold 148 to the one or more fluid outlet lines 152. During the delivery procedure, the one or more valves 136 are positioned such that fluid flow through one or more fluid inlet lines 150 is blocked or closed. In a third position, the one or more valves 136 are oriented such that fluid flow through the one or more fluid inlet lines 150 and the one or more fluid outlet lines 152 or the manifold 148 is blocked or closed. Thus, in the third position, each of the one or more valves 136 isolates the corresponding syringe 132 and prevents fluid flow into and out of the interior volume of the corresponding syringe 132. As such, each of the one or more syringes 132 and the corresponding valve 136 defines a closed system.

The one or more valves 136, fluid inlet lines 150, and/or fluid outlet lines 152 may be integrated into or in fluid communication via the manifold 148. The one or more valves 136 may be selectively positioned to the first, second, and third position by manual or automatic handling. For example, the operator may position the one or more valves 136 into the desired position for filling, fluid delivery, or the closed position. In other examples, at least a portion of the fluid injector system 100 is operable for automatically positioning the one or more valves 136 into a desired position for filling, fluid delivery, or the closed position based on input by the operator or by a protocol executed by the electronic control unit. Suitable fluid injector system mechanisms for automatic positioning of the one or more valves 136 are described in PCT International Publication No. WO 2016/112163.

With continued reference to FIGS. 1 to 3, according to some non-limiting embodiments or aspects, the fluid injector system 100 may have a connection port 192 that is configured to form a releasable fluid connection with at least a portion of the SUDS 190. In some examples, the connection port 192 may be formed on the MUDS 130. Desirably, the connection between the SUDS 190 and the connection port 192 is a releasable connection to allow the SUDS 190 to be selectively connected to and disconnected from the connection port 192. In some examples, the SUDS 190 may be disconnected from the connection port 192 and disposed after each fluid delivery procedure, and a new SUDS 190 may be connected to the connection port 192 for a subsequent fluid delivery procedure. The SUDS 190 may be used to deliver one or more medical fluids to a patient by a SUDS fluid line having a distal end that may be selectively disconnected from the body of the SUDS 190 and connected to a patient catheter. Other examples and features of the SUDS 190 are described in U.S. Patent Publication No. 2016/0331951, the disclosure of which is incorporated herein by reference in its entirety.

Referring again to FIG. 1, the fluid injector system 100 may include one or more user interfaces 124, such as a graphical user interface (GUI) display window. The user interface 124 may display information pertinent to a fluid injection procedure involving the fluid injector system 100, such as injection status or progress, current flow rate, fluid pressure, and volume remaining in the at least one bulk fluid source 120 connected to the fluid injector system 100 and may be a touch screen GUI that allows an operator to input commands and/or data for operation of the fluid injector system 100. The interface 124 may be in electronic communication with an electronic controller 900 or processor 904 to allow a user to input parameters and control the processes of a fluid injection procedure. Additionally, the fluid injector system 100 and/or user interface 124 may include at least one control button 126 for tactile operation by an attendant operator of the fluid injector system 100. The at least one control button 126 may be a graphical part of the user interface 124, such as a touch screen.

Referring again to FIG. 3, in some examples, the fluid outlet line 152 may also be connected to a waste reservoir 156 of the fluid injector system 100, for example through SUDS 190. The waste reservoir 156 is desirably separate from the syringes 132 to prevent contamination. In some examples, the waste reservoir 156 is configured to receive waste fluid expelled from syringes 132 during, for example, a flushing, priming, or preloading operation.

Referring again to FIGS. 2 and 3, the fluid outlet lines 152 may be operatively connected to an outlet air detector 200 configured to detect the presence of one or more air bubbles in a fluid path associated with the fluid outlet lines 152. In other embodiments, the air detector 200 may be operatively connected to tubing of the SUDS 190 to detect the presence of air bubbles in a fluid path associated with the SUDS 190. As shown in FIG. 3, the air detector 200 may be in electrical communication with an electronic controller 900 programmed or configured to operate various components of the fluid injector system 100. In particular, the electronic controller 900 may be programmed or configured to control the pistons 103 associated with the syringes 132 to inject fluid from the syringes 132 to the patient, to prime the MUDS 130 and SUDS 190, to halt an injection in response to unsafe volumes of air detected in system 100, and to perform various other function associated with fluid delivery system 100.

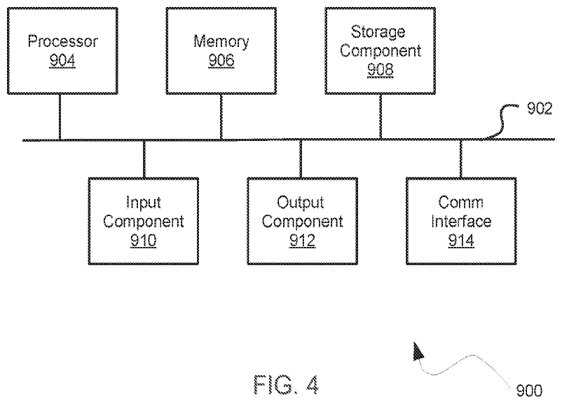

Referring now to FIG. 4, a diagram of example components of the electronic controller 900 for implementing and performing the systems and methods described herein is shown according to embodiments of the present disclosure. In some embodiments, the electronic controller 900 may include additional components, fewer components, different components, or differently arranged components than those shown in FIG. 4. The electronic controller 900 may include a bus 902, at least one processor 904, memory 906, a storage component 908, an input component 910 (such as a GUI, keyboard, or other user interface 124), an output component 912 (such as a GUI or other user interface 124), and a communication interface 914 (such as a GUI or other user interface 124). The bus 902 may include a component that permits communication among the components of the electronic controller 900. In some non-limiting embodiments, the at least one processor 904 may be implemented in hardware, firmware, or a combination of hardware and software. For example, the at least one processor 904 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 906 may include random access memory (RAM), read only memory (ROM), and/or another type of dynamic or static storage device (e.g., flash memory, magnetic memory, optical memory, etc.) that stores information and/or instructions for use by the at least one processor 904.

With continued reference to FIG. 4, the storage component 908 may store information and/or software related to the operation and use of the electronic controller 900. For example, the storage component 908 may include a hard disk (e.g., a magnetic disk, an optical disk, a magneto-optic disk, a solid state disk, etc.) and/or another type of computer-readable medium. The input component 910 may include a component that permits the electronic controller 900 to receive information, such as via user input (e.g., the GUI, a touch screen display, a keyboard, a keypad, a mouse, a button, a switch, a microphone, etc.). Additionally, or alternatively, the input component 910 may include a sensor for sensing information (e.g., a global positioning system (GPS) component, an accelerometer, a gyroscope, an actuator, etc.). The output component 912 may include a component that provides output information from the electronic controller 900 (e.g., the GUI, a display, a speaker, one or more light-emitting diodes (LEDs), etc.). The communication interface 914 may include a transceiver-like component (e.g., a transceiver, a separate receiver and transmitter, etc.) that enables the electronic controller 900 to communicate with other devices, such as via a wired connection, a wireless connection, or a combination of wired and wireless connections. The communication interface 914 may permit the electronic controller 900 to receive information from another device and/or provide information to another device. For example, the communication interface 914 may include an Ethernet interface, an optical interface, a coaxial interface, an infrared interface, a radio frequency (RF) interface, a universal serial bus (USB) interface, a Wi-Fi® interface, a cellular network interface, Bluetooth, and/or the like. The input component 910, output component 912, and/or the communication interface 914 may correspond to, or be components of, the one or more user interfaces 124 (see FIG. 1).

With continued reference to FIG. 4, the electronic controller 900 may perform method described herein based on the at least one processor 904 executing software instructions stored by a computer-readable medium, such as the memory 906 and/or the storage component 908. A computer-readable medium may include any non-transitory memory device. A memory device includes memory space located inside of a single physical storage device or memory space spread across multiple physical storage devices. Software instructions may be read into the memory 906 and/or the storage component 908 from another computer-readable medium or from another device via communication interface 914. When executed, software instructions stored in the memory 906 and/or storage component 908 may cause processor 904 to perform one or more processes described herein. Additionally, or alternatively, hardwired circuitry may be used in place of or in combination with software instructions to perform one or more processes described herein. Thus, embodiments described herein are not limited to any specific combination of hardware circuitry and software. The term “programmed or configured,” as used herein, refers to an arrangement of software, hardware circuitry, or any combination thereof on one or more devices.

Referring again to FIGS. 1 to 3, during preparation and performance of an injection procedure utilizing the fluid injector system 100, air may be unintentionally introduced into various system components. Ideally, such air would be removed by a priming/purging operation of the MUDS 130 and the SUDS 190 subsequent to filling the syringes 132 from the bulk fluid sources 120, as described herein. However, due to various phenomena such as surface adhesion of air bubbles to internal walls of the syringes 132, fluid paths 150, tubing of the SUDS 190, etc., some air may remain in the system components after the priming/purging operation. In addition to being potentially dangerous to the patient, air bubbles injected into the patient's vasculature can cause artifacts to appear in reconstructed images generated by the clinical procedure. Such artifacts may negatively impact quality of the reconstructed images and, consequently, may have a negative impact on patient diagnosis. Even volumes of air that are not physiologically harmful to the patient may nevertheless adversely affect image quality.

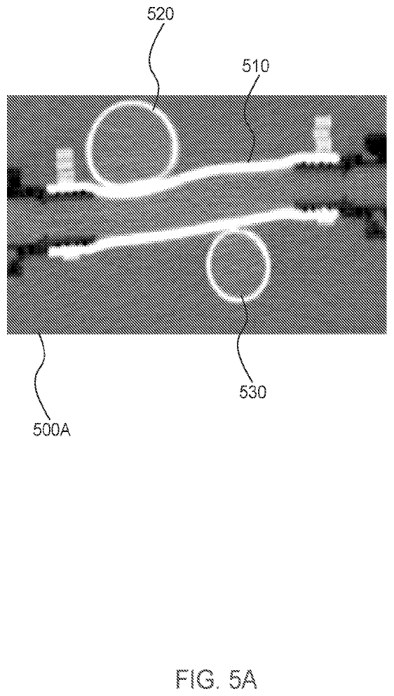

Referring now to FIGS. 5A to 5C, differences in exemplary reconstructed images 500A, 500B, 500C are shown. The images 500A, 500B, 500C shown in FIGS. 5A, 5B, and 5C, respectively, are taken using a testing device simulating a patient's pulmonary artery 510, ascending aorta 520, and descending aorta 530. FIG. 5A shows an image 500A generated by an imaging procedure in which medical fluid containing no appreciable amount of air is injected through the pulmonary artery 510. FIG. 5B shows an image 500B generated using the same imaging procedure as in FIG. 5A, but with an air bubble of approximately 0.1 mL in volume injected with the medical fluid. Injection of the air bubble resulted in a plurality of artifacts 540 occurring in the reconstructed image 500B. In the image 500B, the plurality of artifacts 540 present as curved lines extending inside and outside of the pulmonary artery 510. Such artifacts 540, while not necessarily indicative of a harmful volume of air, nevertheless adversely affect the quality of the image 500B and may result in difficult, incorrect, and/or incomplete diagnosis during review of the image 500B, potentially requiring additional imaging procedures. FIG. 5C shows an image 500C generated using the same imaging procedure as in FIG. 5A, with an air bubble 550 injected with the medical fluid and becoming adhering to a wall of the descending aorta 530. The bubble 550 is readily apparent in the image and, like the artifacts 540 of FIG. 5B, may obstruct the image of the patient's vasculature potentially affecting patient diagnosis. The sensitivity of conventional imaging equipment causes even small air bubbles to be clearly visible in reconstructed images. An air bubble having a volume of 0.1 mL at nominal blood pressure of 120/80 mmHg has a spherical diameter of between 5.5 mm to 5.6 mm in the patient's vasculature. However common CT scanners have a spatial resolution down to 0.5 mm; meaning that an air bubble as small as 0.5 microliters is potentially visible in the reconstructed images 500A, 500B, 500C.

In order to prevent degradation of image quality due to small volume air injections, as shown for example in FIGS. 5B and 5C and to prevent potentially harmful injection of large volumes of air, the fluid injector system 100 may include an air detection device configured to communicate with the controller 900, which may shut down an injection procedure prior to injection of air into the patient and/or alert a user of the presence of one or more air bubbles in the fluid. In some embodiments, the controller 900 may be configured to shut down an injection procedure in response to detection of air in the MUDS 130 and or the SUDS 190. In other embodiments, the controller 900 may be configured to only shut down the injection procedure after a threshold volume of air has been detected in the fluid. The latter configuration has an advantage that small volumes of air which are neither harmful to the patient nor have a large adverse effect on image quality do not cause clinically unnecessary shutdowns of the injection procedure. Unnecessary shut down of an injection procedure can impact workflows, lead to patient and/or operator dissatisfaction with the injection process, and require the patient to be exposed to additional radiation when the aborted injection procedure is re-performed. As such, it is desirable to that the fluid injector system 100 can differentiate between significant and insignificant volumes of air in the fluid injector system 100.

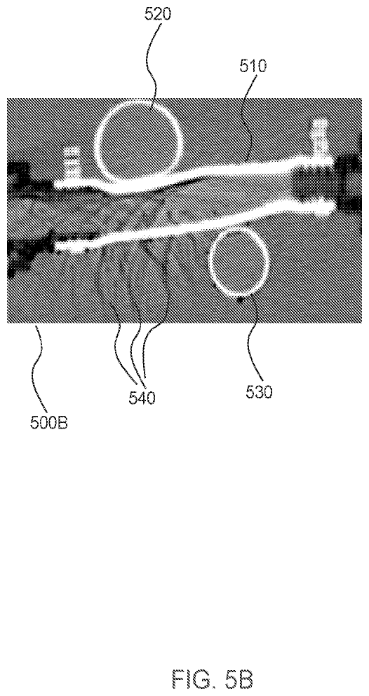

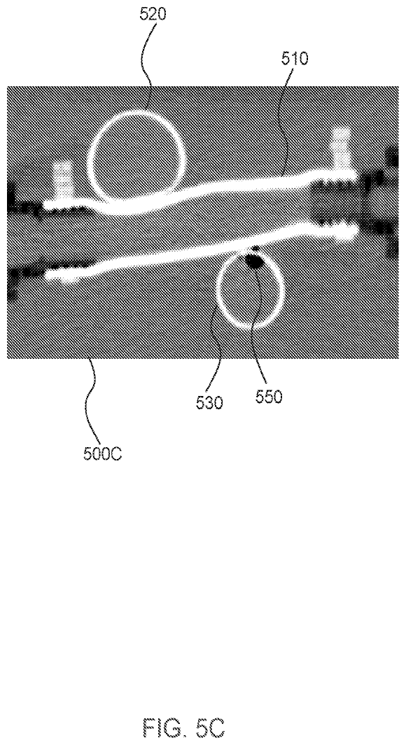

Referring now to FIG. 6, the outlet air detector 200 discussed herein in connection with FIG. 3 is illustrated in operative connection with either the fluid outlet line 152 of the MUDS 130 or tubing of the SUDS 190. The air detector 200 may be configured to detect one or more air bubbles in a fluid path associated with the fluid outlet line 152 or tubing of the SUDS 190. The air detector 200 may define a channel 202 into which the fluid outlet line 152 or the SUDS 190 is inserted. In certain embodiments, the channel 202 may at least partially compress and/or restrict expansion of the fluid outlet line 152 or the SUDS 190 in order to prevent measurement error due to compliance-based swelling of the fluid outlet line 152 or the SUDS 190 within the channel 202. The air detector 200 may be an ultrasonic sensor which uses an acoustic signal to differentiate air from medical fluid (e.g. contrast or saline) in the fluid path. In various embodiments, the air detector 200 may provide digital output. When the air detector 200 is digital, the air detector 200 may output a low voltage electrical signal when one or more air bubbles are detected in the fluid path associated with the air detector 200, and the air detector 200 may output a high voltage electrical signal when medical fluid (with no air or an insignificant amount of air) is detected in the fluid path 152 associated with the air detector 200. FIG. 7 illustrates a graph of output voltage signal against time for a digital air detector during an exemplary fluid injection procedure. The voltage peaks 702 indicate the presence of medical fluid (and absence of air) in the associated fluid path 152, while the low-voltage valleys 704 indicate the presence of one or more air bubbles in the associated fluid path 152. The output voltage signal may be transmitted to and received by electronic controller 900 (see FIG. 4).

While the foregoing description and FIGS. 6 and 7 are generally directed to embodiments in which the air detector 200 is an ultrasonic sensor, the air detector 200 may alternatively be an optical sensor, electromagnetic radiation sensor, motion sensor, or the like and the electronic controller 900 may be programmed or configured to recognize the output signal from any of these types of sensors in order to differentiate air from medical fluid in the fluid path.

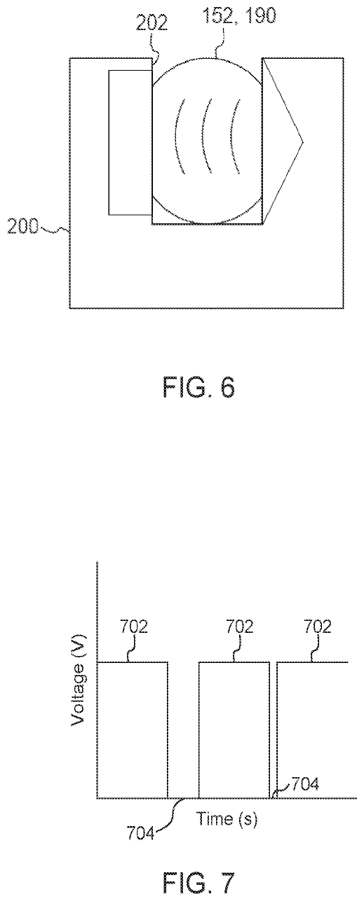

In various embodiment of the present disclosure, the air detector 200 may be used in conjunction with the electronic controller 900 to detect, determine, and respond to a volume of air present in the fluid path during an injection procedure. Referring now to FIG. 8, a method 800 for determining a volume of one or more air bubbles in a fluid path of the fluid injector system 100 is illustrated according to an embodiment of the present disclosure. Generally, the method includes steps for determining the presence of one or more air bubbles and then determining the volume of those one or more air bubbles based at least in part on a calculated fluid flow rate and a calculated fluid pressure within the fluid path. In some embodiments, the method 800 may be a computer-implemented method executed by the at least one processor 904 of the electronic controller 900. In some embodiments, the present disclosure is directed to a computer program product including non-transitory computer readable media having one or more instructions that, when executed be the at least one processor 904, cause the at least one processor 904 to perform the steps of the method 800.

As shown in the sequence diagram of FIG. 8, at step 802 the method 800 may include initiating an injection procedure in which at least one medical fluid is injected into the fluid path associated with the air detector 200. The injection procedure may be initiated by the at least one processor 904 either automatically or in response to a command entered in the fluid injector system 100 by an operator. The injection procedure may be stored as one or more instructions on a non-transitory computer readable media, such as memory, accessible by the at least one processor 904. In certain embodiments, one or more parameters may be entered, adjusted, or changed by a user prior to or during an injection procedure.

Still referring to FIG. 8, at step 804, the method 800 may include receiving an electrical signal from the air detector 200. The electric signal output by the air detector 200 may be received by the at least one processor 904, and the at least one processor 904 may be programmed or configured to determine, based on the electrical signal, whether one or more air bubbles is present in the fluid path associated with the air detector. As described herein, the air detector 200 may output a digital electrical signal from the air detector 200 to the at least one processor 904. As described herein with reference to FIG. 7, the electrical signal may be a low voltage if one or more air bubbles is detected in the fluid path and the air detector 200 is digital. In some embodiments, the at least one processor 904 may be further programmed or configured to identify a lead end and a tail end of each air bubble in the fluid by identifying a change in the digital signal from high to low (indicating the lead end of a bubbles) and a subsequent change in the digital signal from low to high (indicating the tail end of the bubble). Changes in the voltage due to the presence of the one or more air bubbles may vary according to the cross-section of the air bubble. For example, if the air bubble has sufficient volume to substantially fill the cross-section of the fluid path, the measured or observed change in voltage may be greater than if the volume of the air bubble is less than the cross-section of the fluid path (e.g., the bubble diameter is less than the inner diameter of the fluid path).

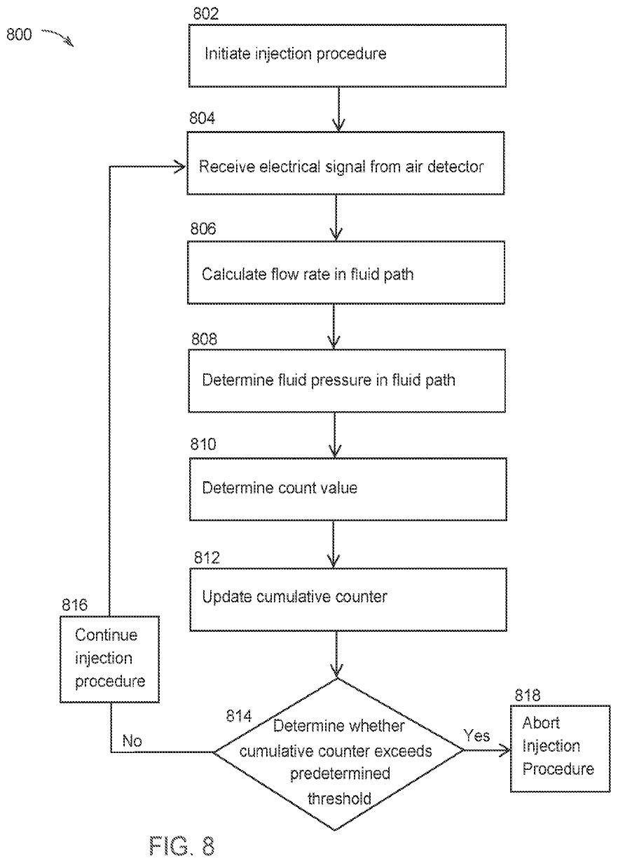

Still referring to FIG. 8, at step 806, the method 800 may include calculating a flow rate in the fluid path. The at least one processor 904 may be programmed or configured with various methodologies for calculating, via measurement and/or estimation, the flow rate in the fluid path. In some embodiments, the at least one processor 904 may calculate the flow rate by measuring the change in the position of the one or pistons 103 within the syringes 132 (as shown in FIG. 3). Based on relative positions of the piston 103 within the syringe 132 over a measured time interval, the at least one processor 904 may calculate the rate at which medical fluid is injected from the syringes 132. In some embodiments, the at least one processor 904 may calculate the flow rate based, at least in part, on a commanded flow rate of the injection procedure. The commanded flow rate is the programmed injection rate for the injection procedure which may be included in one or more instructions stored on stored in the memory 906 and/or the storage component 908 accessible by the at least one processor 904 or may be entered by a user into the at least one processor 904 prior to or during an injection procedure. However, due to capacitance, compliance, or associated mechanical slack of one or more of the components in the fluid injector system 100, the actual flow rate of fluid within the fluid path might not be accurately ascertained from either piston position within the syringe or from the commanded flow rate. A nonlimiting example of a graph illustrating the difference between a commanded flow rate and an actual flow rate over time is shown in FIG. 9. As may be appreciated from FIG. 9, the curve of the actual flow rate 720, for example as measured at the location of the air detector 200, may generally lag behind a curve of the commanded flow rate 730 because some of the fluid pressure is absorbed by system capacitance of components of the fluid injector system 100 instead of being converted in to kinetic fluid flow. The lag may be particularly noticeable during an initial rise time 732 of the commanded flow rate, which may be an approximately vertical portion of the commanded flow rate curve 730 where at least a portion of the pressure applied to the system is converted to system capacitance, due to, for example, pressurized swelling of system components, uptake of mechanical slack, and compression of one or more air bubbles in the fluid. Further, a lag may also be observed at the end of a fluid injection 734 where pressure from the mechanical components, for example the piston, is halted but release of system capacitance causes the fluid to flow. For example, in some experimental injections conducted in accordance with nonlimiting embodiments of the present disclosure, the commanded flow rate may be programmed to reach 6 mL/s within 400 milliseconds of initiating the injection, while the actual flow rate only reaches approximately 0.06 mL/s after 400 milliseconds. According to this embodiment, the actual flow rate at the air detector 220 may not reach the commanded flow rate of 6 ml/s until 2-3 seconds after the injection begins. As such, the actual flow rate may have an error factor of approximately 100 relative to the commanded flow during the first 400 milliseconds of the injection procedure. A similar but inverse effect is observed at the end of a programmed fluid injection where the programmed fluid flow rate may drop to 0 mL/sec within 400 milliseconds but, due to release of stored capacitance, the actual flow rate does not drop to 0 mL/second for 2-3 seconds after piston motion is stopped.

To compensate for the effects of system capacitance on the actual flow rate within the fluid path, the at least one processor 904 may apply one or more correction algorithm to the commanded flow rate to estimate the actual flow rate. The correction algorithm may be derived from one or more equations and/or look-up table containing system and injection parameters stored in the memory 906 and/or storage component 908 accessible by the at least one processor 904. In some embodiments, the one or more correction algorithm may include an equation to reduce the processing power required to convert the commanded flow rate to an estimated actual flow rate. An example of the equation is shown in Equation 1:

Actual flow rate=Commanded flow rate×(1−e^{-t/τ}) Equation 1:

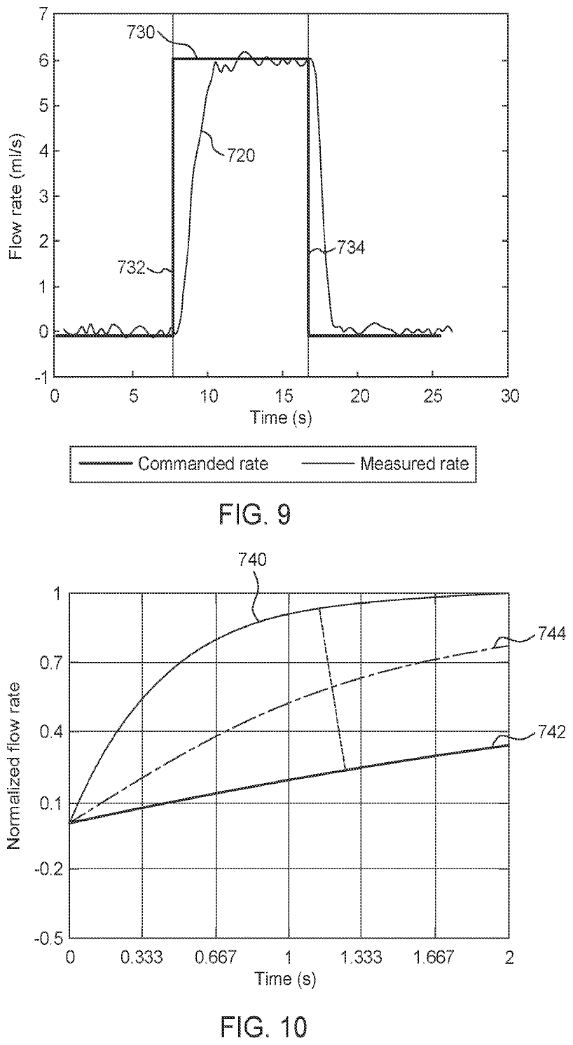

In Equation 1, “Actual flow rate” is the estimated or calculated flow rate taking into account injection parameters and system capacitance in milliliters per second (mL/s), “Commanded flow rate” is the programmed injection rate in mL/s (e.g. the flow rate defined by the curve 730 in FIG. 9), “t” is the time after the injection procedure has started in seconds, and “τ” is a first order time constant. According to various embodiments, “τ” may be determined prior to performing the injection procedure and/or may be stored in the memory 906 and/or the storage component 908 for access by the at least one processor 904 during the injection procedure. In particular, “τ” may be selected to minimize error relative to actual fluid flow rate between various injection procedures. Examples of injection parameters that may affect actual flow rate include, but are not limited to, syringe size and fluid volume, tubing diameter, position of plunger in syringe, syringe and tubing material, commanded flow rate, fluid viscosity, volume of air present, and the like. FIG. 10 illustrates a nonlimiting example of a graph showing the derivation of Equation 1 from an embodiment of an experimental injection procedure. Curve 740 is a normalization of actual flow rate over time for an experimental injection procedure having a relatively short rise time of the commanded fluid flow rate. Curve 742 is a normalization of actual flow rate over time for an experimental injection procedure having a relatively long rise time of the commanded fluid flow rate. Curve 744 represents the flow rate over time as calculated by Equation 1, with the value of “τ” optimized such that curve 744 falls approximately between curves 740 and 742. Thus, the error between curve 744 and curve 740 is approximately equal to the error between curve 744 and 742.

By optimizing “τ” in this manner according to certain embodiments, a single value for “τ” may be used to provide a sufficiently accurate estimation of actual flow rate for a wide variety of commanded flow rates. As will be described herein, the at least one processor 904 may perform a calculation using the optimized value for “τ”. Furthermore using a single optimized value of “τ” can significantly reduce the processing demands on the at least one processor 904 as Equation 1 may be repeatedly calculated in intervals of hundreds of milliseconds throughout the injection procedure and appropriate adjustments made. In some embodiments where available processing power is not of particular concern, the multiple values of “τ” corresponding to various positions of the pistons 103 within the syringes 132 may be used, and the at least one processor 904 may use a stored look-up table to select the appropriate value for “τ” during each calculation of Equation 1. Alternatively or in addition, in some embodiments, the at least one processor 904 may use other known parameters of the injection procedure and/or fluid injector system 100, such as the type of fluid injected, the catheter size, etc., to further select an appropriate “τ” value to enhance accuracy of the flow rate estimation.

Still referring to FIG. 8, at step 808, the method 800 may include determining a fluid pressure in the fluid path. In some embodiments, the at least one processor 904 may determine the fluid pressure in the fluid path by measuring a motor current driving the piston 103 (shown in FIG. 3). Motor current driving the piston 103 is a function of fluid pressure and other known, measurable, or predictable system variables, and therefore the at least one processor 904 may determine the fluid pressure in the fluid path based on measured motor current. In some embodiments, the at least one processor 904 may determine the fluid pressure in the fluid path via one or more pressure sensors, e.g. pressure transducers, directly or indirectly measuring fluid pressure in the fluid path. As with flow rate, system capacitance and other variables may have an effect on the fluid pressure within the fluid path and fluid pressure should be measured once steady state fluid flow is achieved.

Still referring to FIG. 8, at step 810, the method 800 may include determining a count value of the one or more air bubbles detected in the fluid path based on a duration for which the electrical signal from the air detector 200 indicating the presence of the air bubble is received at step 804, the flow rate calculated at step 806, and the fluid pressure determined at step 808. In certain embodiments, the voltage measured by the air detector may also be factored into the count value. The count value may be representative of, or serve as a proxy for, a volume of the one or more air bubbles detected by the air detector. A new count value may be determined each time the air detector 200 samples the fluid path—i.e. each time the air detector 200 measures air in the fluid path and transmits an electrical signal to the at least one processor 904. Using the count value eliminates the need for the actual volume of the one or more air bubbles to be calculated each time the air detector 200 samples the fluid path. Additionally, in certain embodiments, using the count value may allow subsequent calculations to be performed without using decimal values, such that no floating point calculations are required, further reducing the required processing power to execute the method 800.

The count value may be correlated to the volume of the one or more air bubbles according to an equation, such as Equation 2:

Scaled air vol.=Flow rate×Motor const×Count val.×P scalar Equation 2:

In Equation 2, “Scaled air vol.” is the volume of the one or more air bubbles normalized to a predetermined pressure, for example 1 atmosphere (atm). “Count val.” in Equation 2 corresponds to the count value—a time duration for which the air detector 200 detects one or more air bubbles in the fluid path. That is, “Count val.” corresponds to the time duration for which the air detector 200 transmits, and the at least one processor 904 receives, the electrical signal indicating one or more air bubbles are present in the fluid path (typically a low voltage signal if the air detector 200 is digital). “Count val.” may use units selected to minimize processing power demand on the at least one processor 904. For example, the units of “Count val.” may be selected such that 1 mL of air at 1 atm corresponds to a count value of 45×10⁶. While the time duration associated with “Count val.” may be useful, “Count val.” may not provide a complete representation of the volume of the one or more air bubbles without correcting for fluid flow rate and fluid pressure in the fluid path. As such, “Flow rate” and Pressure scalar “(P scalar”) factors are introduced in Equation 2 to correct for the fluid flow rate and fluid pressure in the fluid path.

First addressing the correction for flow rate, flow rate in the fluid path must be accounted for because the time duration for which the air detector 200 senses the presence of the one or more air bubbles in the fluid path is directly correlated to the velocity of the one or more bubbles. For example, if the one or more air bubbles flow through the fluid path at 10 mL/s, the time duration for which the air detector 200 senses the one or more air bubbles will be ten times as long as if the one or more air bubbles flow through the fluid path at 1 mL/s (assuming the same fluid pressure at both 10 mL/s and 1 mL/s). “Flow rate” in Equation 2 may correspond to the fluid flow rate calculated in step 806, in units of mL/s. In some embodiments, Equation 2 may further include Motor constant (“Motor const”) which is a constant selected based on the programming of the motors driving the pistons 103 (see FIG. 3). In particular, “Motor constant” is selected based on how the motors receive the commanded flow rate 730 (see FIG. 9) from the at least one processor 904. In some embodiments, “Motor constant” is selected such that Equation 2 may be calculated by the at least one processor 904 using only integer values and not floating values. By using only integer values in the calculation, the processing power required to calculate Equation 2 may be reduced. In some embodiments, Motor constant may a unit-less constant be between approximately 1 and 10,000, and in some embodiments may be approximately 30.

Next addressing the correction for fluid pressure, fluid pressure must be accounted for because the time duration for which the air detector 200 senses the presence of the one or more air bubbles in the fluid path is directly correlated to the fluid pressure within the fluid path. That is, due to the compressibility of air, a bubble of air will have a greater volume at a low fluid pressure whereas to the same amount of air will have a lower volume at a higher fluid pressure. Approximating air in the fluid path as an ideal gas, the relationship between pressure and volume of the one or more air bubbles is described by the ideal gas law as P₁V₁=P₂V₂. As an example of this principle, an air bubble having a volume of 1 mL at 1 atm has a volume of only 0.05 mL at 20 atm. For a fluid path having an internal diameter of 2.24 millimeters (0.088 inches), a 1 mL air bubble at 1 atm would occupy approximately 254 mm (10 inches) of fluid path length, whereas the same air bubble at 20 atm would occupy approximately 13 millimeters (0.5 inches) of fluid path length. Thus, the time duration for which the air detector 200 senses the air bubble would be approximately 20 times longer at 1 atm than and 20 atm (assuming the same flow rate at both 1 atm and 20 atm). To address the effect of fluid pressure on the duration for which the air detector 200 senses the one or more air bubbles, “Pressure scalar” in Equation 2 may correspond to the fluid pressure determined in step 808, modified according to Equation 3:

$$Pressure scalar = ( 1 + fluid pressure in atm ) \times ( \frac{1}{atm} ) \qquad Equation 3$$

In Equation 3, “Pressure scalar” is the same “Pressure scalar” from Equation 2, and “fluid pressure in atm” is the fluid pressure in the fluid calculated at step 806, in units of atm. The term “(1/atm)” in Equation 3 makes the calculated “Pressure scalar” unit-less.

Using Equations 1 to 3 described herein, the count value may be determined and adjusted to account for flow rate and fluid pressure within the fluid path each time the air detector 200 samples the associated fluid path and transmits an electrical signal to the at least one processor 904 indicating the presence of an air bubble. Each time the transmitted electrical signal indicates the presence of one or more air bubbles in the fluid path, steps 804 through 810 of the method 800 may be repeated and a new count value for the additionally detected one or more air bubble may be determined. According to various embodiments, the sampling rate of the air detector 200 may be selected to balance processing power demand on the at least one processor and accuracy of the count value determination (and ultimately accuracy of air volume calculation). A higher sampling rate of the air detector 200 may increase volume calculation accuracy but require more processing power, as steps 804 to 810 will be repeated at a higher frequency. Conversely, a lower sampling rate may reduce accuracy volume calculation accuracy but require less processing power.

Still referring to FIG. 8, at step 812, the method 800 may include updating a cumulative counter with the count value of the each additionally detected one or more air bubbles, as determined at step 810 over the course of a fluid injection procedure. The cumulative counter may be representative of a cumulative volume of air that has passed through the fluid path during the injection procedure. Each time a new count value is determined at step 810 in response to detection of air in the fluid path, the determined count value from 810 is added to the cumulative counter in 812. Prior to initiating the injection procedure, the cumulative counter may be set to zero, representing that no amount of air has passed through the fluid path. In some embodiments, each time the cumulative counter is updated at step 812, the at least one processor 904 may determine whether the cumulative counter exceeds a predetermined threshold at step 814. In some embodiments, the predetermined threshold may be representative of a volume of air predetermined to be below a threshold volume of air considered safe to inject into a patient. In some embodiments, the predetermined threshold may be selected based on patient characteristics such as age, weight, etc. that may affect the threshold volume of air that can be safely injected into the patient. In some embodiments, the predetermined threshold may be representative of a volume of air known to cause undesirable imaging artifacts (as shown, for example, in FIGS. 5B and 5C) during image reconstruction. In some embodiments, the predetermined threshold may be stored in the memory 906 and/or the storage component 908 accessible by the at least one processor 904. In some embodiments, the predetermined threshold may be entered by an operator via the one or more user interfaces 124 (see FIG. 1) prior to the start of an injection procedure or may be determined by the at least one processor 904 based at least in part on one or more injection parameters and patient characteristic entered by the operator.

In some embodiments, the predetermined threshold may be scaled in the same manner as the count value discussed in step 810 such that the cumulative counter can be directly compared to the predetermined threshold without converting the cumulative counter to actual volume of the one or more bubbles. For example, as discussed herein in connection with step 810 and Equation 2, the count value may be normalized such that a count value of 45×10⁶ correspond to 1 mL of air at 1 atm. Likewise, the predetermined threshold associated with the cumulative counter may be set in the same units as the count value to allow the cumulative counter can be compared directly to the predetermined threshold. For example, if the predetermined threshold is intended to correspond to a volume of air equal to 1 mL at 1 atm, the value of the predetermined threshold may be set to 45×10⁶, equal to the count value for 1 mL of air at 1 atm

Still referring to FIG. 8, at step 814, if the cumulative counter is below the predetermined threshold, the injection procedure may continue by path 816, and steps 804 through 814 may be repeated until the injection procedure is completed of the cumulative counter exceeds the predetermined threshold. Conversely, at step 814, if the cumulative counter exceeds the predetermined threshold, the injection procedure may be aborted at step 818, for example, by halting movement of the pistons 103 within the syringes 132 (see FIG. 3) and/or by closing one or more valves, such as the valves 136, associated with the fluid path to prevent an further fluid and air injection to the patient. Halting an injection procedure in this manner may prevent an unsafe or otherwise undesirable volume of air being delivered to the patient.

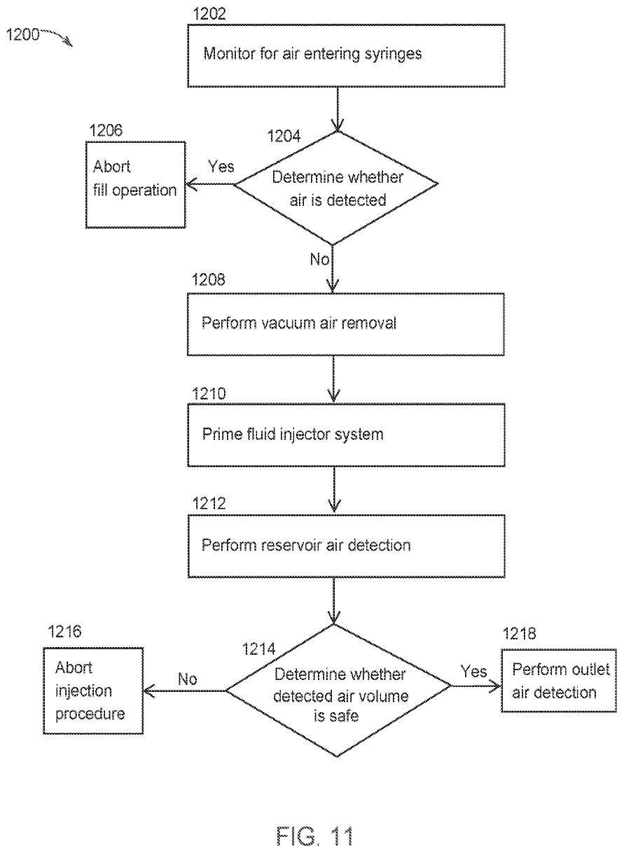

In some embodiments, the method 800 may be employed as part of a more comprehensive air detection scheme for preventing injection air into the patient. Referring now to FIG. 11, a method 1200 of air detection and mitigation method is illustrated according to an embodiment of the present disclosure. The method 1200 may be initiated by the at least one processor 904 either automatically or in response to a command entered in the fluid injector system 100 by an operator. In some embodiments, the method 1200 may be stored as one or more instructions on a non-transitory computer readable media, such as memory, accessible by the at least one processor 904.

At step 1202, the method 1200 may include monitoring for air entering the syringes 132 during a fill operation. The primary mechanism by which air may enter the syringes 132 (and more generally the MUDS 130 in the case of the fluid injector system 100 illustrated in FIG. 1-3) occurs during the syringe filling process, in which reverse movement of the piston 103 draws fluid from the bulk fluid sources 120 into the syringes 132. If the bulk fluid sources 120 run empty or are not properly fluidly connected to the syringes 132, air may be drawn into the syringes 132. At step 1202 of the method 1200, any air bubbles entering the syringes 132 by way of the bulk fluid sources 120 may be detected by one or more inlet air detectors 210 in operative communication with one or more of the fluid inlet lines 150, for example of the MUDS 130 (see FIG. 3). In some embodiments, the one or more inlet air detectors 210 may be optical sensors tuned to differentiate between the presence of air and fluid in the fluid inlet lines 150. Further details of the structure and function of inlet air detectors are described, for example, in International Application Publication No. WO 2019/204605, the disclosure of which is hereby incorporated by reference in its entirety.

Referring again to FIG. 11, at step 1204, the method 1200 may include determining whether air is detected by the inlet air detectors 210. The at least one processor 904 may be programmed or configured to determine whether air is present in the inlet lines 150 based on the electrical signal transmitted by the inlet air detectors 210. In other embodiments, the presence of air in the one or more syringes may be determined as described in International Application Publication No. WO 2019/204605. If significant volumes or only air is detected during filling of the syringes, the fill operation may be aborted at step 1206 and an alert provided to the user to check the fluid volume of the bulk fluid container and replace the container, if necessary. In some embodiments, the electronic controller 900 may display an alert message via the one or more interfaces 124 indicating that air has been detected. If small volumes or no air is detected by the inlet air detectors 210 and the filling operation of the syringes 132 is completed, the method may proceed to step 1208.

With continued reference to FIG. 11, at step 1208, the method 1200 may include performing vacuum air removal from the syringes 132. The vacuum air removal process may be used to remove air bubbles that adhere to internal surfaces of the syringes 132 during filling of the syringes 132 from the bulk fluid sources 120. Such air bubbles may have strong enough adherence to the syringes 132 that typical priming operations have insufficient flow rates to dislodge the air bubbles. For example, air bubbles adhering to internal surfaces of the syringes 132 might not be dislodged by merely advancing the pistons 103 to force fluid out of the syringes 132. The vacuum air removal process of step 1208 may include closing the valves 136 to create a closed system within each of the syringes 132 and retracting the pistons 103 to create a vacuum (negative pressure) in each of the syringes 132. The vacuum created in the syringes 132 causes air bubbles adhered to surfaces of the syringes 132 to expand, dislodge from the surfaces of the syringes 132, and float to the top of the syringes 132 where the air bubbles can be evacuated by a subsequent priming operation of step 1210. Further details regarding the vacuum air removal process are described in International Application Publication No. WO 2019/204617, the disclosure of which is hereby incorporated by reference in its entirety.

Referring still to FIG. 11, the method 1200 may include, at step 1210, priming the fluid injector system 100 in preparation to inject a patient with fluid. Priming the fluid injector system 100 may be performed, for example, by injecting fluid from the syringes 132 through the manifold 148, fluid path 152, and SUDS 190 to dislodge any remaining air bubbles and eject the dislodged air bubbles into the waste reservoir 156 or another waste receptacle. In some embodiments, the electronic controller 900 may be programmed or configured to automatically inject a predetermined volume of fluid from the syringes 132 during the priming operation. In some embodiments, the electronic controller 900 may be programmed or configured to automatically inject fluid from the syringes 132 until no air is detected by the outlet air detector 200. Further details of priming operations are described in International Application Publication Nos. WO 2018/144369, and WO 2020/046929, the disclosures of which are hereby incorporated by reference in their entireties.

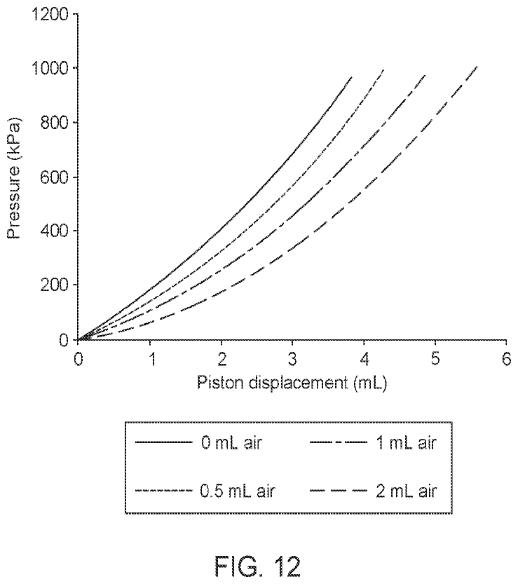

With continued reference to FIG. 11, at step 1212, the method 1200 may include performing reservoir air detection of the syringes 132 to detect air caused by defects, such as cracks, in disposable components of the fluid injector system 100 such as the MUDS 130 or air that has not been purged or primed out of the system by previous steps 1208, 1210. For example, defects in the system components may introduce more air into the syringes 132 than can be reasonably purged during the priming operation of step 1212 such that performing an injection procedure may be unsafe. Reservoir air detection may also provide redundancy in case the inlet air detectors 210 fail to detect the presence of air at step 1202. Performing reservoir air detection at step 1212 may include closing the valves 136 to create a closed system within each of the syringes 132 and advancing the pistons 103 to generate a target pressure in the syringes 132, for example 1000 kPa or greater. The position of the pistons 103 within the syringes 132 may be monitored along with pressure and compared to a calibration data set which contains plunger displacement and pressure values for a reservoir that is completely free of air. FIG. 12 illustrates an example data set of plunger displacement and pressure values for 0 mL, 0.5 mL, 1 mL, and 2 mL or air in the syringes 132. As may be appreciated from FIG. 12, an increase in air in the syringes 132 requires additional piston displacement to reach the target pressure due to the compressibility of air, see e.g., International Application Publication No. WO 2019/204605.

In some embodiments, performing reservoir air detection at step 1212 may include calculating a volume of air in each of the syringes 132. The volume of air in each of the syringes 132 may be calculated according to Equation 4:

$${V}_{a i r} = \frac{Δ V ( {P}_{a t m} + {P}_{g} )}{{P}_{g}} \qquad Equation 4$$

In Equation 4, “Vₐᵢᵣ” is the volume of air in the syringe 132, “ΔV” is the change in volume required to pressurize the syringe 132 to the target pressure, “Pₐₜₘ” is the atmospheric pressure, and “P_{g}” is the target pressure.

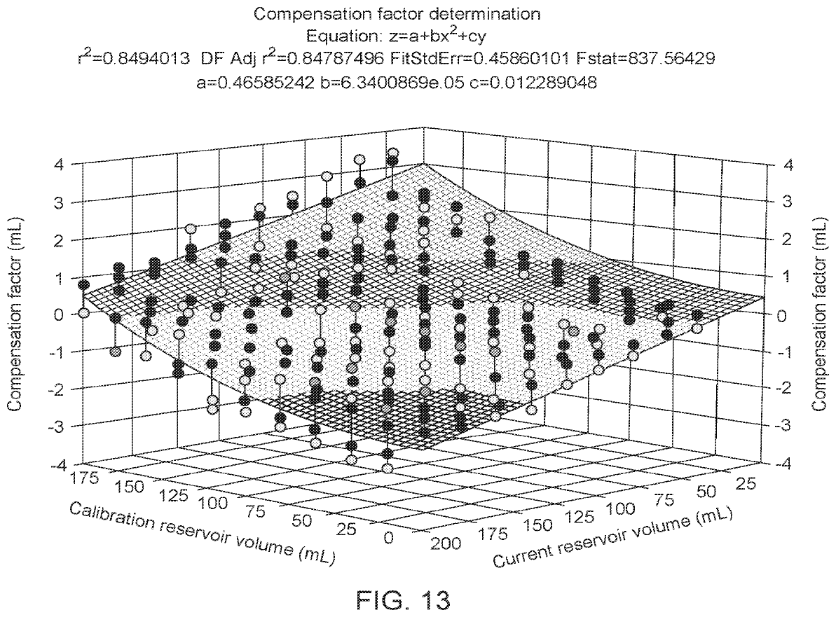

In certain embodiments, calculating the air volume according to Equation 4 may be confounded by compliance of the syringes 132. Compliance may be asymmetric depending on the internal volume of the syringes 132 and pressure generated in the syringes 132 during step 1212. For example, under a pressure of 1000 kPa, a syringe 132 originally filled to a maximum capacity of 200 mL may experience an internal volume increase of 6 mL due to compliance-induced swelling. Conversely, if the same syringe 132 is filled to only 10 mL, a pressure of 1000 kPa may increase the internal volume by capacitance swelling by less than 2 mL. As such, the piston displacement and pressure data for a syringe 132 containing 4 mL of air when originally filled to 10 mL will be indistinguishable from the same data collected from a syringe originally filled to 200 mL with no air. To account for this source of error, a compensation algorithm may be applied to Equation 4 to adjust the calculated air volume depending on the position to which the syringe 132 is filled. An example of a compensation algorithm is illustrated graphically in FIG. 13, which provides a 3D equation for determining a compensation factor based on the current volume of the syringe 132 being assessed.

Referring again to FIG. 11, the method 1200 may include, at step 1214, determining whether the volume of air in each syringe 132 calculated at step 1212 is unsafe or otherwise unsuitable for performing an injection procedure. If the volume of air in any of the syringes 132 is unsafe, for example, if the volume of air could potentially harm the patient if injected or may cause significant image defects, the injection procedure may be aborted at step 1216. In some embodiments, the electronic controller 900 may display an alert message on the one or more interfaces 124 indicating that air has been detected. If no air or a safe volume of air is present in the syringes 132, the method may proceed to step 1218.

Referring still to FIG. 11, the method 1200 may include, at step 1218, performing outlet air detection during the injection procedure. Step 1218 may include monitoring the fluid outlet lines 152 and/or tubing of the SUDS 190 for one or more air bubbles during the injection procedure, substantially the same as described in steps 802-818 of the method 800 discussed herein. In its entirety, the method 1200 provides for air detection at various locations of the fluid injector system 100 during various stages of the preparation and performance of an injection procedure, thus providing an air detection scheme ensuring unsafe volumes of air, and/or air volumes likely to interfere with image reconstruction, are not injected into a patient.

To analyze and quantify the efficacy of air bubble detection of the method 1200 described herein, the fluid injection system 100 was attached to a test device and various fluid injection procedures were performed. The test device was attached to the distal end of the SUDS 190 such that the test device received medical fluid injections in the same manner as the vasculature of a patient during a clinical imaging procedure. The test device included an air trap specifically designed to collect and subsequently measure the volume of all air delivered from the SUDS 190 during the simulated injection procedures. Prior to performing the simulated injection procedures, the test device was validated by injecting and measuring known quantities of air to ensure that overall uncertainty in the air volume measurement was sufficiently small to draw accurate conclusions from the results of the experimental injection procedures.

Across thirty experimental injection procedures conducted using the test device, there were zero instances of visible (to the human eye) air bubbles in the SUDS 190 prior to the injection being performed. As such, it can be reasonably interpreted that the vacuum air removal at step 1208 of the method 1200 and the subsequent priming operation at step 1210 of the method 1200 were effective at purging air from the system 100 prior to the injection being performed. Further, across the 30 experimental injection procedures, the average injected air volume was 0.005 mL±0.006 mL with a maximum volume of 0.017 mL. It is noted that in none of the thirty experimental injection procedures did the fluid injector system 100 stop the injection procedure in response to detection of air bubbles. Given this evidence, in combination with the average measured air volume within the expected error distribution of the testing device, it can be reasonably interpreted that the fluid injector system 100 did not inject a detectable amount of air throughout the simulated clinical use. These findings suggest that the air detection and removal of method 1200 as described herein successfully eliminates the injection of harmful air bubbles during simulated clinical use.

While various examples of the present disclosure were provided in the foregoing description, those skilled in the art may make modifications and alterations to these examples without departing from the scope and spirit of the disclosure. For example, it is to be understood that features of various embodiments described herein may be adapted to other embodiments described herein. Accordingly, the foregoing description is intended to be illustrative rather than restrictive. The disclosure described hereinabove is defined by the appended claims, and all changes to the disclosure that fall within the meaning and the range of equivalency of the claims are to be embraced within their scope.

1. A method for determining a volume of one or more air bubbles in a fluid path of a fluid injector system, the method comprising:
initiating an injection procedure in which at least one medical fluid is injected into the fluid path;
receiving an electrical signal from an air detector of the fluid injector system, wherein the electrical signal indicates a presence of one or more air bubbles in the fluid path;
calculating a flow rate of fluid in the fluid path;
determining a fluid pressure in the fluid path;
determining a count value of the one or more air bubbles based on a duration for which the electrical signal is received, the flow rate, and the fluid pressure, wherein the count value is representative of the volume of the one or more air bubbles; and
updating a cumulative counter with the count value of the one or more air bubbles, wherein the cumulative counter is representative of a cumulative volume of air that has passed through the fluid path during the injection procedure.

2. The method of claim 1, further comprising halting the injection procedure in response to the cumulative counter exceeding a predetermined threshold.

3. The method of claim 1, further comprising continuing the injection procedure in response to the cumulative counter being below a predetermined threshold.

4. The method of claim 2, wherein the predetermined threshold is programmed into a memory of the fluid injector system.

5. The method of claim 1, wherein calculating the flow rate in the fluid path comprises estimating an actual flow rate in the fluid path based on:
a commanded flow rate for the injection procedure; and
a compliance of one or more components of the fluid injector system.

6. The method of claim 1, further comprising setting the cumulative counter to zero prior to initiating the injection procedure.

7. The method of claim 1, further comprising purging the one or more air bubbles from the fluid injector system prior to initiating the injection procedure.

8. A fluid injector system comprising:
at least one syringe configured for injecting at least one medical fluid;
a fluid path in fluid communication with the at least one syringe;
an air detector configured to detect one or more air bubbles in the fluid path;
at least one processor programmed or configured to:
initiate an injection procedure in which the at least one medical fluid is injected from the at least one syringe into the fluid path;
receive an electrical signal from the air detector, wherein the electrical signal indicates a presence of one or more air bubbles in the fluid path;
calculate a flow rate of fluid in the fluid path;
determine a fluid pressure in the fluid path;
determine a count value of the one or more air bubbles based on a duration for which the electrical signal is received, the flow rate, and the fluid pressure, wherein the count value is representative of a volume of the one or more air bubbles; and
update a cumulative counter with the count value of the one or more air bubbles, wherein the cumulative counter is representative of a cumulative volume of air that has passed through the fluid path during the injection procedure.

9. The fluid injector system of claim 8, wherein the at least one processor is further programmed or configured to halt the injection procedure in response to the cumulative counter exceeding a predetermined threshold.

10. The fluid injector system of claim 8, wherein the at least one processor is further programmed or configured to continue the injection procedure in response to the cumulative counter being below a predetermined threshold.

11. The fluid injector system of claim 9, wherein the predetermined threshold is programmed into a memory of the fluid injector system.

12. The fluid injector system of claim 8, wherein calculating the flow rate in the fluid path comprises estimating an actual flow rate in the fluid path based on:
a commanded flow rate for the injection procedure; and
a compliance of one or more components of the fluid injector system.

13. The fluid injector system of claim 8, wherein the at least one processor is further programmed or configured to set the cumulative counter to zero prior to initiating the injection procedure.

14. The fluid injector system of claim 8, wherein the at least one processor is further programmed or configured to purge the one or more air bubbles from the fluid injector system prior to initiating the injection procedure.

15. A computer program product for determining a volume of one or more air bubbles in a fluid path of a fluid injector system, the computer program product comprising: non-transitory computer readable media comprising one or more instructions that, when executed by at least one processor of the fluid injector system, cause the at least one processor to:
initiate an injection procedure in which at least one medical fluid is injected into the fluid path;
receive an electrical signal from an air detector of the fluid injector system, wherein the electrical signal indicates a presence of one or more air bubbles in the fluid path;
calculate a flow rate of fluid in the fluid path;
determine a fluid pressure in the fluid path;
determine a count value of the one or more air bubbles based on a duration for which the electrical signal is received, the flow rate, and the fluid pressure, wherein the count value is representative of the volume of the one or more air bubbles; and
update a cumulative counter with the count value of the one or more air bubbles, wherein the cumulative counter is representative of a cumulative volume of air that has passed through the fluid path during the injection procedure.

16. The computer program product of claim 15, wherein the one or more instructions further cause the at least one processor to halt the injection procedure in response to the cumulative counter exceeding a predetermined threshold.

17. (canceled)

18. The computer program product of claim 16, wherein the predetermined threshold is programmed into a memory of the fluid injector system.

19. The computer program product of claim 15, wherein calculating the flow rate in the fluid path comprises estimating an actual flow rate in the fluid path based on:
a commanded flow rate for the injection procedure; and
a compliance of one or more components of the fluid injector system.

20. The computer program product of claim 15, wherein the one or more instructions further cause the at least one processor to set the cumulative counter to zero prior to initiating the injection procedure.

21. The computer program product of claim 15, wherein the one or more instructions further cause the at least one processor to purge the one or more air bubbles from the fluid injector system prior to initiating the injection procedure.
